SEQUENCE LISTING (1 sequence)

SEQ ID NO: 1 - DNA, 1588 bp, synthetic construct
  source 1..1588: mol_type = other DNA
taatacgactcactatagggagaccacaacggtttccctctagaaataattttgtttaactttaagaaggagatatacatatggactataaagacgatgacgataaacatatgggcagcatggataaagattgcgaaatgaaacgtaccacactggatagtccgctgggtaaactggaactgagcggttgtgaacagggtctgcatgaaattaaactgttaggtaaaggcaccagcgcagcagatgcagttgaagttccggcaccggcagcagttttaggtggtccggaaccgctgatgcaggcaaccgcatggctgaatgcatattttcatcagccggaagccattgaagaatttccggttcctgcactgcatcatccggtttttcagcaagaaagctttacccgtcaggttctgtggaaactgctgaaagttgttaaatttggcgaggtgattagctatcagcagctggcagcactggcaggtaatccggcagcaaccgcagcagttaaaaccgcactgagcggcaatccggttccgattctgattccgtgtcatcgtgttgttagcagcagcggtgcagttggtggttatgaaggtggtctggcagttaaagaatggctgctggcacatgaaggccatcgtctgggcaaacctggtttaggcggtagcagtggtgcaagccctgcagcaccggctccggcaagtccagcagcaccagcacctagcgcaccagccggtggtgatatcaaactgcagcagagcggtgcagaactggcacgtccgggtgcaagcgttaaaatgagctgtaaaaccagcggttatacctttacacgttataccatgcattgggttaaacagcgtcctggtcaaggtctggaatggattggttatatcaatccgagccgtggttataccaattacaaccagaaattcaaagacaaagcaaccctgaccaccgataaaagcagcagcaccgcctatatgcagctgagcagcctgaccagcgaagatagcgcagtttattactgtgcacgctattatgatgatcactattgcctggattattggggtcagggcaccacactgaccgttagcagcgttgaaggtggtagcggtggttcaggtggtagtggcggtagtggtggtgttgatgatattcagctgacccagagtccggcaattatgagcgcaagtccgggtgaaaaagttaccatgacctgtcgtgccagcagcagcgttagctatatgaattggtatcagcagaaaagcggtacaagcccgaaacgttggatttatgataccagcaaagttgcaagcggtgttccgtatcgttttagcggtagcggttcaggcaccagctatagtctgaccattagctcaatggaagcagaagatgcagcaacctattattgtcagcagtggtcaagcaatccgctgacctttggtgcaggtacaaaactggaactgaaataagtcgaccggctgctaacaaagcccgaaaggaagctgagttggctgctgccaccgctgagcaataactagcataaccccttggggcctctaaacgggtcttgaggggttttttg

(19) United States
(12) Patent Application Publication
(10) Pub. No.: US 2026/0345814 A1
(43) Pub. Date: Nov. 5, 2026

(54) METHODS FOR LEVERAGING LIPID NANOCARRIER PHASE TRANSITIONS

(71) Applicant: Northwestern University, Evanston, IL (US)

(72) Inventors: Neha Prashant Kamat, Evanston, IL (US); Sara Carina Fedosejevs, Evanston, IL (US)

(21) Appl. No.: 19/450,253

(22) Filed: Jan. 15, 2026

Related U.S. Application Data
(60) Provisional application No. 63/746,157, filed on Jan. 16, 2025.

(51) Int. Cl. A61K 47/69; A61K 35/17; A61K 38/17; A61K 40/11; A61K 40/31; A61P 37/06

(52) U.S. Cl. CPC A61K 47/6911; A61K 35/17; A61K 38/1774; A61K 40/11; A61K 40/31; A61P 37/06

(57) ABSTRACT

Disclosed herein is a method of activating a cell with a lipid nanocarrier. The method includes determining a melting transition temperature of the lipid nanocarrier, selecting an incubation temperature, and contacting the cell with the lipid nanocarrier at the incubation temperature, wherein the melting transition temperature and the incubation temperature differ by no greater than about 10 °C.

### CROSS-REFERENCE TO RELATED APPLICATIONS

The present application claims priority to U.S. Provisional Patent Application No. 63/746,157 that was filed Jan. 16, 2025, the entire contents of which are hereby incorporated by reference.

### STATEMENT REGARDING FEDERALLY SPONSORED RESEARCH

This invention was made with government support under grant number DMR-2308691 awarded by the National Science Foundation. The government has certain rights in the invention.

### REFERENCE TO AN ELECTRONIC SEQUENCE LISTING

The content of the electronic sequence listing (70258102756.xml; Size: 3,374 bytes; and Date of Creation: Jan. 14, 2026) is herein incorporated by reference in its entirety.

### FIELD OF THE DISCLOSURE

The disclosed technology is generally directed to surface-modified lipid nanocarriers. More particularly the technology is directed to melting transition temperature of lipid nanocarriers.

### BACKGROUND

Surface-modified lipid nanocarriers are increasingly used as artificial antigen-presenting cells for therapeutic applications in immunotherapy. Within these nanocarriers, the role of the lipids is typically limited to providing structure/stability of the particle, to anchoring a targeting moiety, and/or to altering the biodistribution of the nanocarriers in vivo. However, lipid membranes also possess special thermodynamic properties that impact their function.

For example, surface-modified lipid nanocarriers include artificial antigen-presenting cells (aAPCs). Artificial antigen-presenting cells that target and activate T cells, either ex or in vivo, have become an important approach for T cell immunotherapy. Among the various nanoparticles used to design aAPCs, lipid-based nanoparticles are a popular choice due to their high biocompatibility and low systemic toxicity in vivo. While several design principles have been identified that enhance the biodistribution and compatibility of lipid-based particles in vivo, there remain a surprisingly low number of approved aAPC therapies for the clinic. This dearth of approved therapies for immune-cell activation suggests a greater need for improved lipid nanocarriers and strategies for using them that robustly and efficiently activate their target cells.

Accordingly, there is a need in the field for compositions for activating cells and methods of using the same.

### SUMMARY

In an aspect, provided herein is a method of activating a cell with a lipid nanocarrier, the method comprising: determining a melting transition temperature of the lipid nanocarrier; selecting an incubation temperature, wherein the melting transition temperature and the incubation temperature differ by no greater than about 10 °C; and contacting the cell with the lipid nanocarrier at the incubation temperature. In embodiments, the melting transition temperature and the incubation temperature differ by no greater than about 5.0 °C. In embodiments, the melting transition temperature and the incubation temperature differ by no greater than about 1.0 °C. In embodiments, the melting transition temperature and the incubation temperature are substantially the same.

The lipid nanocarrier may comprise a lipid selected from the group consisting of 1,2-dioleoyl-sn-glycero-3-phosphocholine (DOPC), 1,2-di-(9Z-octadecenoyl)-sn-glycero-3-phospho-L-serine (sodium salt) (18:1PS;DOPS), 1,2-dilauroyl-sn-glycero-3-phospho-L-serine (sodium salt) (12:0PS; DLPS), 1,2-dimyristoyl-sn-glycero-3-phospho-(1′-rac-glycerol) (sodium salt) (14:0PG; DMPG), 1,2-dimyristoyl-sn-glycero-3-phosphocholine (14:0PC; DMPC), 1-stearoyl-2-myristoyl-sn-glycero-3-phosphocholine (18:0-14:0PC), 1-myristoyl-2-palmitoyl-sn-glycero-3-phosphocholine (14:0-16:0PC), 1,2-dimyristoyl-sn-glycero-3-phospho-L-serine (sodium salt) (14:0PS; DMPS), 1,2-dielaidoyl-sn-glycero-3-phosphoethanolamine (18:1(A9-Trans)PE), 1-myristoyl-2-stearoyl-sn-glycero-3-phosphocholine (14:0-18:0PC), 1,2-dipalmitoyl-sn-glycero-3-phospho-(1′-rac-glycerol) (sodium salt) (14:0 PG; DPPG), 1,2-dipalmitoyl-sn-glycero-3-phosphocholine (16:0PC;DPPC), 1,2-diheptadecanoyl-sn-glycero-3-phosphocholine (17:0 PC), 1,2-dipalmitoyl-sn-glycero-3-phospho-L-serine (sodium salt) (16:0 PS; DPPS), 1,2-distearoyl-sn-glycero-3-phospho-(1′-rac-glycerol) (sodium salt) (18:0 PG, DSPG), and any combinations thereof.

The lipid nanocarrier may comprise about 70 mol % of a lipid.

The lipid nanocarrier may comprise cholesterol and 1,2-distearoyl-sn-glycero-3-phosphoethanolamine-N-[methoxy(polyethylene glycol)-2000](ammonium salt) (18:0 PE PEG 2000).

The lipid nanocarrier may comprise a lipid anchor. The lipid anchor may comprise polyethylene glycol (PEG). The lipid anchor may comprise 1,2-dioleoyl-sn-glycero-3-phosphoethanolamine-N-[benzylguanine(polyethylene glycol)-2000](18:1 PE PEG BG).

The lipid anchor may be functionalized with at least one surface tag. The at least one surface tag may be selected from the group consisting of an antibody, a protein, a co-stimulatory ligand, a cytokine, an antigen, and any combinations thereof. The at least one surface tag may comprise αCD3.

The lipid nanocarrier may comprise a liposome.

The cell may be a T-cell.

The incubation temperature may be between about 4 °C and about 65 °C. In embodiments, the incubation temperature is between about 20 °C to about 55 °C.

In another aspect, provided herein is a method of treating a T-cell related disorder in a subject in need, the method comprising: selecting a lipid nanocarrier comprising a melting transition temperature, wherein the melting transition temperature and the subject's body temperature differ by no greater than 10 °C; and administering an effective amount of the lipid nanocarrier to the subject.

The method may further comprise administering an effective amount of a chimeric antigen receptor (CAR) T-cell to the subject in need. The method may further comprise determining the melting transition temperature of the lipid nanocarrier prior to the step of selecting the lipid nanocarrier.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure will be better understood and features, aspects, and advantages other than those set forth above will become apparent when consideration is given to the following detailed description thereof. Such detailed description makes reference to the following drawings.

FIGS. 1A-IC show a schematic overview of the assembly of lipid-based artificial antigen presenting cells (aAPCs) and activation of Jurkat cells. Panel (A) shows aAPCs are assembled from large unilamellar vesicles (LUVs) containing 70% phospholipids, 29% cholesterol, 0.9% PEG-lipids, and 0.1% benzyl guanine (BG)-modified lipids, in which % refers to mol %. BG is a substrate for the self-labelling protein SNAP-tag and allows for a SNAP-tag-αCD3 scFv fusion protein to be conjugated to LUVs via attachment of SNAP-tag to the BG groups on the LUV surface. These particles are incubated overnight at 37 °C with an engineered Jurkat cell line expressing GFP upon activation. Activation is confirmed via microscopy and quantified with flow cytometry. This assay is based on the one-pot approach of LUV assembly introduced previously. Panel (B) shows the standard liposome formulation contains 18:1 PC (DOPC, Tₘ=−17 °C) keeping the liposome in the liquid disordered phase during interaction with the membrane of Jurkat cells. Phospholipids with a Tₘ>T_{activ} form higher ordered membranes in the liquid ordered phase at T_{activ} when mixed with cholesterol. Panel (C) shows phase states in lipid bilayers are associated with distinct membrane properties such as compressibility, heat capacity or diffusivity, density and permeability. Higher ordered states, such as the gel phase or cholesterol-induced liquid ordered phase are more rigid and display lower diffusivity rates than the liquid disordered state. During the transition between two phases, membrane fluctuations are maximized and diverging physical properties and nonlinear behavior emerges. Inventors hypothesize that this nonlinear behavior should promote membrane rearrangement and corresponding activation in target Jurkat cells.

FIGS. 2A-2D show liposome composition determines efficiency of Jurkat cell activation. Panel (A) shows flow cytometry analysis of activated Jurkat cells determined by the number of GFP-positive cells after incubation with large unilamellar vesicles (LUVs). DOPC LUVs lacking CD3 (−CD3,+LUV) result in ~1% activation of Jurkat cells, similar to the % activation after incubation with unconjugated αCD3 only (+CD3, −LUV) or buffer (−CD3; −LUV). Addition of Phorbol 12-myristate 13-acetate (PMA) and Ionomycin activates the cells and is used as a positive control for the flow cytometry analysis. Functionalized liposomes (here: DOPC conjugated to αCD3) activate 20-50% of the cell population. GFP positive cells are determined via flow cytometry. Panel (B) shows a comparison of activation efficiencies for treatment with LUVs assembled from lipids with three different headgroups and two different chain lengths reveals no correlation of Jurkat cell activation with either parameter. LUVs based on PC lipids show lower activation potential for the investigated chain lengths compared to PS and PG LUVs. While LUVs with PS (14:0) perform far better than PS (16:0) (+70%), for PG LUVs this trend is reversed. Panel (C) shows screening of LUVs arranged according to ascending theoretical melting temperatures of the respective lipid type alone. Incubation temperature (T_{activ}) relative to Tₘ determines the state of the lipid membrane (neglecting the influence of cholesterol and medium). % Jurkat cells activated follows a parabolic curve as a function of LUV Tm with maximal activation occurring for LUVs that have a Tₘ close to T_{activ}. Panel (D) shows results for the activation efficiencies of Jurkat cells with different LUVs as function of the lipid Tₘ they are based on. Data is divided into three headgroups and different chain lengths yield different Tₘ values. Relative activation refers to the difference in activation efficiency compared to reference LUVs based on 18:1PC. R Data represents mean of at least 3 biological replicates type with error bars representing the standard error of mean (SEM).

FIGS. 3A-3C show measurements of generalized polarization (GP) of Laurdan to determine the Tₘ of 16:0 PC in panel (A) 14:0 PS in panel (B) and 16:0 PG in panel (C) based LUVs. From left to right: 100% PL in PBS, activation formulation in PBS, activation formulation in full RPMI. For each condition GP values (light grey) are plotted as function of temperature and the phase transition temperature is identified as the temperature with greatest GP change corresponding with a maximum in the pointwise derivative of the GP values with respect to the temperature (black). For pure phospholipid liposomes the measured Tₘ is in good agreement with the literature values and similar holds for the addition of cholesterol and PEGylated lipids to the LUVs. In RPMI media in which the activation studies are conducted the melting of PG and PS based LUVs is affected due to their interaction with divalent ions in the media and the Tₘ differs from their literature value. For 14:0 PS (panel B) LUVs the shape of the GP function varies significantly as a nonlinear change in the slope is present, marked with an arrow. An inflection point of the derivative is found at T_{inflict}=37.7 °C and is increased compared to the transition temperature in PBS. Fluorescence intensity was measured with a spectrophotometer and GP values and their pointwise derivative were calculated with python.

FIGS. 4A-4C show Jurkat activation efficiency is a function of the proximity of LUV Tm to the temperature of activation studies T_{act}iv. Panel (A) shows Jurkat activation data now plotted in ascending order of the corrected LUV Tₘ values from left to right using the Tₘ, values determined in the previous section. Additional grey (white to black) scaling visualizes the proximity of the Tₘ to the temperature at which the activation was investigated (37 °C). Activation levels are enhanced when |Tₙ-T_{activ}| is small and the LUVs are close to their melting transition. Except for 16:0 PG LUV, the activations levels for LUVs in their L_{O} phase (Tₘ>37 °C) are overall lower compared to the lipids at a similar distance to 37 °C in melting transition in the L_{D} phase. Panel (B) shows exemplary direct comparison of activation of Jurkat cells incubated with different PC-based LUVs as a function of LUV Tₘ measured at 30 °C (top) and 37 °C (bottom). For both measurements, the same batch of liposomes and conjugated antibody was used. At T_{activ}=30 °C, 18:0-14:0PC LUVs (data point at Tₘ=30 °C) and 14:0PC LUVs (data point at Tₘ=24 °C) activated the most cells while at T_{activ}=37 °C 14:0-16:0 LUVs (Tₘ=35 °C) activated the most cells. This trend demonstrates the correlation between activation efficacy of a LUV and the proximity of its Tₘ to the temperature of activation. Data points present the mean of 2 technical replicates with error bars presenting the standard error of mean (SEM). Panel (C) shows pairwise comparison of LUVs with two different melting temperatures (Tₘ₁ and Tₘ₂, with Tₘ₁<Tₘ2, denoted as Tₘ₁/Tₘ₂ on the x axis) and their activation efficiencies of Jurkat cells at two different temperatures T_{activ}. Each bar represents the ratio of cells activated with lipid LUV1 to cells activated with LUV2 (% activation with Tₘ₁/% activation with Tₘ₂) at either 30 °C (dark grey) or 37 °C (light grey) to visualize temperature dependent differences in performance. For all pairs the ratio decreases towards T_{activ}=37 °C demonstrating the increasing activation efficiency of the lipid with the higher Tₘ. Measurements were performed n>3 with error bars representing the standard error of mean (SEM).

FIGS. 5A-5D show confocal microscopy images of Jurkat cell activation. Panel (A) shows DPPG giant unilamellar vesicles (GUVs) (right) display dark spots indicating phase separation in the membrane at room temperature in contrast to DMPS (left). Panel (B) shows brightfield image of Jurkat cells accumulating around and adhering to GUVs modified with αCD3. Panel (C) shows analysis of NR12S emission ratio (I₁/I₂, where I₁=565 nm-595 nm and I₂=600 nm-630 nm) in three investigated GUV compositions and in Jurkat cells. An increased r NR12S ratio increases corresponds to increased order in the membrane. Results are the mean of n>10 GUVs/cells with error bars representing the standard error of mean (SEM). Panel (D) shows exemplary ratiometric images for Jurkat cells incubated with DOPC, DMPS, and DPPG GUVs. Each pixel represents the ratio of the two wavelength channels (I₁/I₂). Color bar ranges from purple (lowest ratio) to red (highest ratio). The cutoff values are chosen individually for optimal visibility of ratio differences as the absolute numbers vary with the GUV compositions which are reported in panel (C). Areas of contact between GUVs and Jurkat cells are marked with an arrow indicating shifted NR12S intensity ratios. Next to the DPPG ratio image an excerpt of the intensity weighted scan is shown to highlight bright areas on the GUV surface close to the interaction site with the Jurkat cell (immunological synapse). All scale bars display 10 μm.

FIGS. 6A-6B show Laurdan GP as function of temperature for 18:1 (Δ9-Trans) PE liposomes in their composition similar to the activation study (including 29 mol % cholesterol). Melting transition measured in PBS is shown in panel (A) and RPMI is shown in panel (B).

FIG. 7 shows results for activation at 30 °C normalized to 14:0-18:0 PC. Data set includes PC lipids and the DPPG replicates (relative activation: 1.61). Comparing only PC lipids the maximal activation is shifted to 14:0-16:0PC (Tm=30 °C), but overall DPPG is the best activating sample contrary to the trend.

FIG. 8 shows Activation of Jurkats with functionalized GUVs via αCD3-conjugation after overnight incubation. GFP expression upon activation of Jurkat reporter cell line is visible in microscopy images in all three tested conditions.

### DETAILED DESCRIPTION

In a first aspect, disclosed herein is a method of activating a cell with a lipid nanocarrier. The method includes determining a melting transition temperature of the lipid nanocarrier, selecting an incubation temperature, and contacting the cell with the lipid nanocarrier at the incubation temperature. Inventors remarkably determined that cell activation is enhanced when the melting transition temperature and the incubation temperature are commensurate.

The method disclosed herein includes lipid nanocarriers. The lipid nanocarriers may be used as artificial antigen presenting cells (aAPCs). An “antigen presenting cell” is a cell that displays an antigen bound by major histocompatibility complex (MHC) proteins on its surface for recognition by T cells. APCs are important for adaptive immune response and contribute to immune responses against both intracellular and extracellular pathogens. As used herein, the term “artificial antigen presenting cell” refers to an engineered systems that display antigens and mimic the function of natural APCs in stimulating T cell responses. aAPCs allow for controlled activation and expansion of functional, pathogen- or tumor-specific T cells. Lipid-based nanocarriers with a fluid membrane have been developed as aAPCs to replicate interactions between natural APCs and T cells in nature.

As used herein, the term “lipid nanocarrier” refers to a nanoscale structure composed of lipids. Lipid nanocarriers may include a variety of structures. For example, lipid nanocarrier structures may include one or more lipid bilayers, solid lipid cores, or mixed lipid matrices. For example, lipid nanocarriers may include liposomes, solid lipid nanoparticles, nanostructured lipid carriers, lipid-polymer hybrid nanoparticles, micelles, lipid nanoemulsions. In some cases, the lipid nanocarriers include liposomes. In some cases, the liposomes include vesicles such as giant unilamellar vesicles (GUVs), large unilamellar vesicles (LUVs), and any combinations thereof.

The lipid nanocarriers disclosed herein include at least one dimension ranging from about 0.5 nanometers to about 1000 nanometers, from about 50 nanometers to about 250 nanometers, from about 50 nanometers to about 500 nanometers, from about 50 nanometers to about 150 nanometers. In some cases, the lipid nanocarriers disclosed herein include at least one dimension of about 100 nanometers. In some cases, the at least one dimension is a diameter.

The lipid nanocarriers may contain a variety of components, including lipids. In some cases, the lipids may be selected from the group consisting of 1,2-distearoyl-sn-glycero-3-phosphoethanolamine-N-[methoxy(polyethylene glycol)-2000](18:0 PE PEG 2000), cholesterol, 1,2-dioleoyl-sn-glycero-3-phosphocholine (DOPC), 1,2-di-(9Z-octadecenoyl)-sn-glycero-3-phospho-L-serine (18:1PS;DOPS), 1,2-dilauroyl-sn-glycero-3-phospho-L-serine (12:0PS; DLPS), 1,2-dimyristoyl-sn-glycero-3-phospho-(1′-rac-glycerol) (14:0PG; DMPG), 1,2-dimyristoyl-sn-glycero-3-phosphocholine (14:0PC; DMPC), 1-stearoyl-2-myristoyl-sn-glycero-3-phosphocholine (18:0-14:0PC), 1-myristoyl-2-palmitoyl-sn-glycero-3-phosphocholine (14:0-16:0PC), 1,2-dimyristoyl-sn-glycero-3-phospho-L-serine (14:0PS; DMPS), 1,2-dielaidoyl-sn-glycero-3-phosphoethanolamine (18:1(Δ9-Trans)PE), 1-myristoyl-2-stearoyl-sn-glycero-3-phosphocholine (14:0-18:0PC), 1,2-dipalmitoyl-sn-glycero-3-phospho-(1′-rac-glycerol) (14:0 PG; DPPG), 1,2-dipalmitoyl-sn-glycero-3-phosphocholine (16:0PC;DPPC), 1,2-diheptadecanoyl-sn-glycero-3-phosphocholine (17:0 PC), 1,2-dipalmitoyl-sn-glycero-3-phospho-L-serine (16:0 PS; DPPS), 1,2-distearoyl-sn-glycero-3-phospho-(1′-rac-glycerol) (18:0 PG, DSPG), any salts thereof, and any combinations thereof.

In some cases, the lipid nanocarrier comprises about 50 mol %, about 60%, about 70 mol %, about 80 mol %, about 90 mol % of phospholipid.

In some cases, the lipid nanocarrier disclosed herein may further include cholesterol. In some cases, the lipid nanocarrier comprises about 20 mol %, about 25%, about 30 mol %, about 35 mol %, about 40 mol % of cholesterol. In some cases, the lipid nanocarriers include about 29 mol % cholesterol.

In some cases, the lipid nanocarrier disclosed herein may further include a lipid anchor. As used herein, the term “lipid anchor” refers to a molecule configured to connect a surface tag to the lipid nanocarrier. In some cases, the lipid anchor includes a terminal reactive moiety, such as benzylguanine, for functionalization with a surface tag. Other terminal reactive moieties are suitable, including reactive moieties which are used in amine-reactive chemistries (e.g., such as N-hydroxysuccinimide (NHS) ester or isothiocyanate and amines), thiol reactive chemistries (e.g., maleimide or haloacetyl with thiols), carboxyl-reactive chemistries (e.g., carbodiimide in EDC/NHS and amines), ‘Click’ chemistries (e.g., azide-alkyne cycloaddition, strain promoted azide-alkyne cycloaddition), aldehyde/ketone reactive chemistries (e.g., hydrazone/oxime formation), photo-crosslinking (e.g., diazirine or benzophenone), or an enzyme-mediated conjugation.

In some cases, the lipid anchor may include polyethylene glycol (PEG). In some cases, the lipid anchor may include 1,2-dioleoyl-sn-glycero-3-phosphoethanolamine-N-[benzylguanine(polyethylene glycol)-2000](18:1 PE PEG BG). In some cases, the lipid nanocarrier comprises about 0.2 mol %, about 0.3%, about 0.5 mol %, about 0.8 mol %, about 0.9 mol %, about 1.0 mol %, about 1.2 mol %, about 1.5 mol %, about 1.8 mol %, about 2.0 mol %, of lipid anchor.

In some cases, the lipid nanocarrier further includes at least one surface tag. As used herein, the term “surface tag” refers to a molecule that helps trigger a response in a cell exposed to the lipid nanocarrier. In some cases, a lipid anchor is functionalized with the at least one surface tag. In some cases, the lipid anchor is functionalized terminally with a surface tag. The at least one surface tag includes a cell-specific moiety that is used as an antigen recognized by the cell, such as a T cell-specific antigen. Cell-specific moiety may include antibodies, protein or peptide antigens, cytokines, etc. In some cases, the cell-specific moiety comprises an anti-CD3 antibody (αCD3) and/or an anti-CD2 antibody (αCD2). The αCD3 may be an anti-CD3 scFv. The αCD2 may be an anti-CD2 scFv. In some cases, the cell-specific moiety may be a tumor-associated antigen. The at least one surface tag may include a major histocompatibility complex (MHC), also called the human leukocyte antigen (HLA), which is loaded with the cell-specific moiety. The MHC complex may be a class I MHC for activating cytotoxic T cells (CD8 cells). The MHC complex may be a class II MHC for activating helper T cells (CD4 cells). The at least one surface tag may comprise a co-stimulatory ligand. Co-stimulatory ligands include, but are not limited to CD80, CD86, ICOS-L, anti-CD28, anti-4-1BB ligands, anti-OX40, and anti-CD40.

In some cases, the lipid nanocarriers may further include 1,2-distearoyl-sn-glycero-3-phosphoethanolamine-N-[methoxy(polyethylene glycol)-2000](18:0 PE PEG 2000). In some cases, the lipid nanocarrier may include at least one phospholipid, cholesterol, and 1,2-distearoyl-sn-glycero-3-phosphoethanolamine-N-[methoxy(polyethylene glycol)-2000](18:0 PE PEG 2000).

In some cases, the lipid nanocarrier is designed to deliver an agent, such as a compound, a nucleotide, a peptide, or other bioactive molecule, to trigger a response in a cell exposed to the lipid nanocarrier. In some cases, the response may be targeting a particular site in a subject in need, such as a cell, organelle, tissue, organ, or organ system.

In embodiments, the method includes the step of determining a melting transition temperature (Tm) of the lipid nanocarrier. As used herein, the “melting transition temperature” refers to a temperature at which a material undergoes a phase transition from a more ordered state to a less ordered state. Determining a melting transition temperature may be accomplished by any suitable method, including calorimetric methods or spectroscopic methods. For example, suitable methods may include differential scanning calorimetry, ultraviolet/visible absorbance, fluorescence spectroscopy, circular dichroism, generalized polarization spectroscopy, dynamic light scattering, infrared spectroscopy, Raman spectroscopy, turbidity, optical density, or any combination of techniques. Determining a melting transition temperature may also include referring to references already known in the art, including referencing D. Marsh, Handbook of lipid bilayers, CRC press, 2nd edn., 2013, which is incorporated by reference herein.

The method further includes selecting an incubation temperature. As used herein, the term “incubation temperature” refers to a temperature at which a biological, chemical, or physical process is maintained for a period of time to achieve a desired outcome. In some cases, the incubation temperature ranges from about 4 °C to about 100 °C, from about 4 °C to about 65 °C, or from about 20 °C to about 55 °C.

In some cases, the melting transition temperature and the incubation temperature differ by no greater than about 10 °C, no greater than about 5.0 °C, no greater than about 1.0 °C. In some cases, the melting transition temperature and the incubation temperature are substantially the same. As used herein, the term “substantially the same” refers to a difference between the lipid melting transition temperature and the incubation temperature of less than 1.0 °C, less than 0.5 °C, less than 0.1 °C.

The method further includes contacting a cell with the lipid nanocarrier at the incubation temperature. In some cases, the cell includes an epithelial cell, a connective tissue cell, a muscle cell, a nervous system cell, a blood cell, an immune cell, a germ cell, or any combinations thereof. In some cases, the cell is in vivo, ex vivo, in vitro, in situ, or in utero. In some cases, the immune cell is an innate immune cell, an adaptive immune cell, a Memory T cell, a Memory B cell, or an innate lymphoid cell. In some cases, the cell is a leukocyte. In some cases, the cell is a lymphocyte, macrophage, or neutrophil. In some cases, the cell is a T-cell. In some cases, the cell is a CAR T cell.

In a second aspect, disclosed herein is a method of treating a T-cell related disorder in a subject in need. The method includes selecting a lipid nanocarrier having a melting transition temperature, wherein the melting temperature and the subject's body temperature differ by no greater than 10 °C; and administering an effective amount of the lipid nanocarrier to the subject.

In some embodiments, the method further includes administering an effective amount of a chimeric antigen receptor (CAR) T-cell therapy to the subject in need. The CAR T cell may be administered at before, after, or at the same time as the lipid nanocarrier.

The terms “chimeric antigen receptor” (“CAR”), “chimeric T cell receptor”, “artificial T cell receptor”, and “chimeric receptor” refer to a polypeptide that binds to cell membrane and has a pre-defined binding domain operably linked to an intracellular signaling domain, where the binding domain has specificity to a target antigen operably and the signaling domain activates the cell when the antigen is bound. More particularly, CARs are engineered receptors, which graft an antigen specificity onto a cytotoxic cell, for example T cells, NK cells or macrophages. For example, CAR proteins are engineered to give T cells the new ability to target a specific protein. This particularly includes receptors wherein the extracellular domain and the cytoplasmic domain are not naturally found together on a single receptor protein. Further, the chimeric receptor is different from the T cell receptor expressed in the native T cell lymphocyte. CARs may comprise an extracellular domain with at least one antigen specific targeting region, a transmembrane domain TM, and an intracellular domain (ID) including one or more co-stimulatory and/or signaling domains in a combination that is not naturally found together on a single protein. The transmembrane (TM) domain is the portion of the CAR that traverses the cell membrane. The TM domain may comprise a hydrophobic alpha helix. In exemplary embodiments, the CD19 CAR comprises a CD8a transmembrane domain. Other TM domains comprise all or a portion of the TM domain of CD28.

The intracellular domain of CARs may comprise additional signaling domains, e.g., portions of proteins that are important for downstream signal transduction. In exemplary embodiments, the CD19 CAR comprises a 4-1BB and a CD3ζ signaling domains. Other signaling domains include, but are not limited to CD28, ICOS, CD27, CD40, OX40 (CD134), and Myd88 signaling domains. Methods of making CARs, expressing them in cells, e.g., T-cells, and utilizing the CAR-expressing T-cells in therapy, are known in the art. See, e.g., International Patent Application Publication Nos. WO2014/208760, WO2014/190273, WO2014/186469, WO2014/184143, WO2014180306, WO2014/179759, WO2014/153270, U.S. Application Publication Nos. US20140369977, US20140322212, US20140322275, US20140322183, US20140301993, US20140286973, US20140271582, US20140271635, US20140274909, European Application Publication No. 2814846, each of which are incorporated by reference in their entirety.

In embodiments the lipid nanocarrier comprises a surface tag that is recognized by the CAR T cell.

The method may further include determining the melting transition temperature of the lipid nanocarrier prior to the step of selecting the lipid nanocarrier. Determining the melting transition temperature of the lipid nanocarrier may include any of the methods described elsewhere herein.

As used herein, “subject” or “a subject in need” refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process. As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a cell or subject by any suitable route for delivery of the therapeutic agent to the desired location.

As used herein, “preventative,” “preventing,” “prevent” and the like refer to partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

As used herein, a “T-cell related disorder” refers to a disease or disorder that involves reduced T cell expression or activation, as well as a disease or disorder that can be alleviated by activation of T cells. In some cases, the subject in need is experiencing an immunodeficiency, an autoimmune disorder, a T-cell malignancy, an infectious pathology, and any combination thereof.

The subject's body temperature is the body temperature of the subject at the time of administering the lipid nanocarrier. In embodiments, the subject's body temperature is about 37 °C.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent.

The terms “effective amount” or “therapeutically effective amount” refer to an amount sufficient to effect beneficial or desirable biological and/or clinical results. The amount of composition (i.e., lipid nanocarrier, CAR T cell therapy) that is therapeutically effective may vary depending on the particular pathogen or the condition of the subject. Appropriate dosages may be determined, for example, by extrapolation from cell culture assays, animal studies, or human clinical trials taking into account body weight of the patient, absorption rate, half-life, disease severity and the like. The dosage lies preferably within a range of circulating concentrations that include the ED50 with little or no toxicity. The dosage may vary within this range depending upon the dosage form employed and the route of administration utilized. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The terms “protein” or “polypeptide” or “peptide” are used interchangeably to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

The terms “engineered polynucleotide”, “recombinant polynucleotide”, “genetically engineered polynucleotide”, and “genetically modified polynucleotide” refer to any manipulation of a polynucleotide that results in a detectable change in a naturally occurring polynucleotide, wherein the manipulation includes, but is not limited to, changes in the sequence of the polynucleotide or inclusion of non-naturally occurring nucleotides or nucleosides.

The terms “antibody” and “antibody molecule” are used herein interchangeably and refer to immunoglobulin molecules or other molecules which comprise an antigen binding domain. Antibodies include whole antibodies (e.g., IgG, IgA, IgE, IgM, or IgD), monoclonal antibodies, chimeric antibodies, humanized antibodies, and antibody fragments, including single chain variable fragments (scFv), single domain antibodies, and antigen-binding fragments, genetically engineered antibodies, among others, as long as the characteristic properties (e.g. ability to bind to the protein of interest or variant) are retained.

The term antibody includes “antibody fragments” or “antibody-derived fragments” and “antigen binding fragments” which comprise an antigen binding domain and displays antigen binding function, for example, Fab, Fab′, F(ab′)2, scFv, Fv, dsFv, ds-scFv, Fd, mini bodies, monobodies, and multimers thereof and bispecific antibody fragments. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), (see for instance Bird et al., Science 242, 423-426 (1988) and Huston et al., PNAS USA 85, 5879-5883 (1988)). Such single chain antibodies are encompassed within the term antibody unless otherwise noted or clearly indicated by context.

The terms “polynucleotide,” “polynucleotide sequence,” “nucleic acid,” and “nucleic acid sequence” refer to a nucleotide, oligonucleotide, polynucleotide (which terms may be used interchangeably), or any fragment thereof. A “polynucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

As used herein, the term “nucleic acid” or “polynucleotide” refers to deoxyribonucleic acid (DNA), ribonucleic acid (RNA) and DNA/RNA hybrids. Polynucleotides may be single-stranded or double-stranded. Nucleic acids include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (geRNA), guide RNA, tracRNA, crRNA, sgRNA, plus strand RNA (RNA(+)), minus strand RNA (RNA(−)), synthetic RNA, genomic DNA (gDNA), PCR amplified DNA, complementary DNA (cDNA), synthetic DNA, or recombinant DNA.

As used herein, the terms “compound”, “small molecular therapeutic agent”, “small molecule compound”, and “small molecule drug” refer to a chemical compound or pharmaceutically acceptable salt thereof having a therapeutic effect and/or enhancing the therapeutic effect of an immunotherapy. Small molecule drugs are typically comprised of 20 to 100 atoms and have a molecular mass of less than 1000 g/mol or 1 kilodalton [kDa]. Small-molecules drugs can typically be administered by a variety of routes (including orally) and can pass through cell membranes to reach intercellular targets.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus <10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

To better improve the design of aAPCs, it is useful to consider the sequence of events that T cells undergo during activation by an antigen-presenting cell. Specifically, for T lymphocytes, there have been a number of studies uncovering the complex processes that occur in the plasma membrane during activation. Upon binding APC antigens, an immunological synapse is formed between the T cell and antigen-presenting cell, triggering a downstream signaling cascade activated by the influx of Ca²⁺ ions. The formation of the immunological synapse is accompanied by a complex reorganization of the T cell membrane, referred to as “bullseye formation”, during which the T cell receptors (TCRs) cluster in the center of the bullseye. The asymmetry of the membrane in this region is reduced as phosphatidylserine (PS) lipids are relocated to the outer leaflet, creating a negative charge sink at the synapse and a local increase in lipid order. Simultaneously, local deformations in the T cell membrane are necessary to bring antigens on the aAPC and receptors on the T cell close enough to bind and initiate this process, requiring local areas with increased elasticity. Altogether, the activation of T cells requires rapid remodeling and reorganization of lipids and proteins on the T cell surface.

The dynamic rearrangements on the T cell membrane during synapse formation and activation suggest that the properties of the aAPCs facilitating this reorganization should enhance the efficiency of synapse formation and T cell activation. Along these lines, several properties of aAPCs have been shown to be important for T cell activation, including the biochemical identity and presentation of antigens and co-stimulatory molecules, as well as physical characteristics of the APCs and aAPCs themselves. Toward the former feature, the density of activating antibodies, such as the commonly used anti-CD3 (TCR stimulus) and anti-CD28 (costimulatory cue), has been shown to impact T cell activation. Toward the latter feature, it has been found that the size, shape, and membrane fluidity of the aAPC can impact aAPC efficacy. For example, the rigidity of the aAPC membrane is an important parameter for the activation process. Since TCRs cluster during activation, a corresponding mobility of the activating antigen on the antigen-presenting cell is advantageous. Indeed, spatial clustering of the targeting moiety has been shown to enhance its binding efficiency to cellular targets.

While previous work has largely focused on the role of static physical properties of aAPCs in activating immune cells, perhaps a more important feature of aAPCs is their capacity to undergo dynamic changes in their properties. Lipid membranes and heterogenous cell membranes are able to undergo phase transitions, also known as melting transitions. Depending on their environment (temperature, pH, pressure, salt concentration, etc.), lipids adopt distinct orientations and phases within a bilayer, ranging from a less dense, disordered chain orientation (liquid disordered phase, L_{D}) to a more crystalline, highly ordered one (gel phase, L_{O}). Synthetic lipid membranes assembled from mixtures of lipids can undergo melting transitions between phase states. At these transition points, the membrane structure becomes highly dynamic, exhibiting large intrinsic fluctuations. The fluctuations can lead to unusual or unexpected events, as properties like compressibility, heat capacity, and area expansion coefficient diverge relative to points below or above the phase transition point. While previous studies on lipid membrane transitions have focused on synthetic lipid mixtures, recent studies have shown these transitions exist in biological membranes as well. These phase transitions are expected to play an important role in cooperatively regulating various cellular functions such as transport processes, protein organization, and enzyme activities. For example, a membrane might get very soft at the phase transition point with an associated increased permeability relative to points below and above the transition point, leading to enhanced signal transduction. These prior results point to a potentially important but overlooked property in the design of nanocarrier membranes for activating signaling cascades in a target cell. Specifically, nanocarriers designed with a melting transition close to that of the target cell membrane's transition point might best support dynamic rearrangements of lipids and proteins on both membranes to efficiently drive cell synapse formation and activation.

Here, Inventors investigated the effect of the melting transition temperature of nanocarrier membranes on the activation of T cells. Using an in vitro platform to assemble aAPCs and assess T cell activation, Inventors explored the effect of the nanocarrier melting transition temperature and its proximity to the cell growth temperature on T cell activation. The results point to a new design consideration of surface functionalized, lipid-based aAPCs to effectively promote target cell activation.

### EXPERIMENTAL

Materials. The following lipids were purchased from Avanti Polar Lipids: 1,2-dioleoyl-sn-glycero-3-phosphocholine (DOPC), cholesterol, 1,2-dioleoyl-sn-glycero-3-phosphoethanolamine-N-[benzylguanine(polyethylene glycol)-2000](18:1 PE PEG BG), 1,2-distearoyl-sn-glycero-3-phosphoethanolamine-N-[methoxy(polyethylene glycol)-2000](ammonium salt) (18:0 PE PEG 2000), 1,2-di-(9Z-octadecenoyl)-sn-glycero-3-phospho-L-serine (sodium salt) (18:1PS;DOPS), 1,2-dilauroyl-sn-glycero-3-phospho-L-serine (sodium salt) (12:0PS; DLPS), 1,2-dimyristoyl-sn-glycero-3-phospho-(1′-rac-glycerol) (sodium salt) (14:0PG; DMPG), 1,2-dimyristoyl-sn-glycero-3-phosphocholine (14:0PC; DMPC), 1-stearoyl-2-myristoyl-sn-glycero-3-phosphocholine (18:0-14:0PC), 1-myristoyl-2-palmitoyl-sn-glycero-3-phosphocholine (14:0-16:0PC), 1,2-dimyristoyl-sn-glycero-3-phospho-L-serine (sodium salt) (14:0PS; DMPS), 1,2-dielaidoyl-sn-glycero-3-phosphoethanolamine (18:1(Δ9-Trans)PE), 1-myristoyl-2-stearoyl-sn-glycero-3-phosphocholine (14:0-18:0PC), 1,2-dipalmitoyl-sn-glycero-3-phospho-(1′-rac-glycerol) (sodium salt) (14:0PG; DPPG), 1,2-dipalmitoyl-sn-glycero-3-phosphocholine (16:0PC;DPPC), 1,2-diheptadecanoyl-sn-glycero-3-phosphocholine (17:0PC), 1,2-dipalmitoyl-sn-glycero-3-phospho-L-serine (sodium salt) (16:0PS; DPPS), 1,2-distearoyl-sn-glycero-3-phospho-(1′-rac-glycerol) (sodium salt) (18:0PG, DSPG).

The fluorescent dyes, 2-dimethylamino-6-lauroylnaphthalene (LAURDAN) and 3-((3-((9-(Diethylamino)-5-oxo-5H-benzo[a]phenoxazin-2-yl)oxy)propyl)(dodecyl) (methyl)ammonio)propane-1-sulfonatem (NR12S), were purchased from Fisher Scientific.

DNA preparation and protein expression. The αCD3 scFv-SNAP-tag plasmid was purchased from Twist Bioscience. The plasmid is designed with a T7 promoter and terminator, a ribosomal binding site and a n-terminal FLAG-tag, and SNAP-tag based on a pJL1 vector. The DNA sequence is provided in Table 1. DNA was amplified and purified with the PureLink™ HiPure Plasmid Midiprep Kit (Invitrogen). The protein was cell free expressed with the PURExpress In Vitro Protein Synthesis kit (New England Biolabs) and additional PURExpress Disulfide Bond Enhancers (New England Biolabs). The reaction was assembled on ice with 3.3 nM plasmid DNA and run for 16 h at 30 °C. Afterwards, the protein was stored at 4 °C.

TABLE 1
DNA sequence of αCD3 scFv SNAP-tag protein.

| | |
|---|---|
| αCD3 | TAATACGACTCACTATAGGGAGACCACAACGGTTTCCCTCTAGAAATAATT |
| scFV | TTGTTTAACTTTAAGAAGGAGATATACATATGGACTATAAAGACGATGAC |
| SNAP- | GATAAACATATGGGCAGCATGGATAAAGATTGCGAAATGAAACGTACCAC |
| tag | ACTGGATAGTCCGCTGGGTAAACTGGAACTGAGCGGTTGTGAACAGGGTC |
| | TGCATGAAATTAAACTGTTAGGTAAAGGCACCAGCGCAGCAGATGCAGTT |
| | GAAGTTCCGGCACCGGCAGCAGTTTTAGGTGGTCCGGAACCGCTGATGCA |
| | GGCAACCGCATGGCTGAATGCATATTTTCATCAGCCGGAAGCCATTGAAG |
| | AATTTCCGGTTCCTGCACTGCATCATCCGGTTTTTCAGCAAGAAAGCTTTA |
| | CCCGTCAGGTTCTGTGGAAACTGCTGAAAGTTGTTAAATTTGGCGAGGTGA |
| | TTAGCTATCAGCAGCTGGCAGCACTGGCAGGTAATCCGGCAGCAACCGCA |
| | GCAGTTAAAACCGCACTGAGCGGCAATCCGGTTCCGATTCTGATTCCGTGT |
| | CATCGTGTTGTTAGCAGCAGCGGTGCAGTTGGTGGTTATGAAGGTGGTCTG |
| | GCAGTTAAAGAATGGCTGCTGGCACATGAAGGCCATCGTCTGGGCAAACC |
| | TGGTTTAGGCGGTAGCAGTGGTGCAAGCCCTGCAGCACCGGCTCCGGCAA |
| | GTCCAGCAGCACCAGCACCTAGCGCACCAGCCGGTGGTGATATCAAACTG |
| | CAGCAGAGCGGTGCAGAACTGGCACGTCCGGGTGCAAGCGTTAAAATGAG |
| | CTGTAAAACCAGCGGTTATACCTTTACACGTTATACCATGCATTGGGTTAA |
| | ACAGCGTCCTGGTCAAGGTCTGGAATGGATTGGTTATATCAATCCGAGCCG |
| | TGGTTATACCAATTACAACCAGAAATTCAAAGACAAAGCAACCCTGACCA |
| | CCGATAAAAGCAGCAGCACCGCCTATATGCAGCTGAGCAGCCTGACCAGC |
| | GAAGATAGCGCAGTTTATTACTGTGCACGCTATTATGATGATCACTATTGC |
| | CTGGATTATTGGGGTCAGGGCACCACACTGACCGTTAGCAGCGTTGAAGG |
| | TGGTAGCGGTGGTTCAGGTGGTAGTGGCGGTAGTGGTGGTGTTGATGATAT |
| | TCAGCTGACCCAGAGTCCGGCAATTATGAGCGCAAGTCCGGGTGAAAAAG |
| | TTACCATGACCTGTCGTGCCAGCAGCAGCGTTAGCTATATGAATTGGTATC |
| | AGCAGAAAAGCGGTACAAGCCCGAAACGTTGGATTTATGATACCAGCAAA |
| | GTTGCAAGCGGTGTTCCGTATCGTTTTAGCGGTAGCGGTTCAGGCACCAGC |
| | TATAGTCTGACCATTAGCTCAATGGAAGCAGAAGATGCAGCAACCTATTA |
| | TTGTCAGCAGTGGTCAAGCAATCCGCTGACCTTTGGTGCAGGTACAAAACT |
| | GGAACTGAAATAAGTCGACCGGCTGCTAACAAAGCCCGAAAGGAAGCTG |
| | AGTTGGCTGCTGCCACCGCTGAGCAATAACTAGCATAACCCCTTGGGGCCT |
| | CTAAACGGGTCTTGAGGGGTTTTTTG (SEQ ID NO: 1) |

Liposome preparation. Liposomes were prepared via thin film hydration: 70 mol % of a phospholipid of interest, 29 mol % cholesterol, 0.9 mol % 18:0 PE PEG 2000, and 0.1 mol %, 18:1 PE PEG BG were mixed in a glass vial in chloroform and gently dried under a flow of nitrogen to create a thin film of the lipid mixture. The films were stored in the vacuum oven overnight to remove residual solvent. Films were rehydrated with PBS to a final lipid concentration of 4 mM at 60 °C for 30-60 minutes and vortexed in between. The vesicle suspensions were extruded through 100 nm filters (Whatman Nuclepore polycarbonate membranes) with a Mini-Extruder set (Avanti) with 11 passes through the membrane to form homogeneous large unilamellar vesicles. For functionalization, 4 μl of the αCD3 cell free expression crude mixture was incubated with 4 μl of liposomes for 2 hrs at room temperature to allow for conjugation of αCD3-SNAP-tag to liposomes containing BG-lipids and was afterwards stored at 4 °C.

GUV preparation. GUVs were prepared via spontaneous swelling. Briefly, 60 μl of a 2.5 mM lipid solution was diluted in 200 μl chloroform in a 20 ml glass vial. Under a stream of nitrogen, the chloroform was evaporated and a thin film formed under constant rotation by hand of the glass. The vial was placed in a vacuum oven for 2 h to remove residual solvent. 2 ml sucrose solution (295 mosm) was gently pipetted into the vial and placed in a 60 °C oven overnight. Without further vortexing of the lipid suspension, 1 ml of the volume was taken out the next morning. To concentrate the GUVs 200 μl of the suspension was carefully pipetted into 1 ml glucose (295 mosm). After 20-30 min, 20 μl of the bottom fraction was collected and transferred to a new tube to either add the GUVs directly to an imaging dish for quality control or to further conjugate the GUVs with 4 μl of the αCD3 antibody fragment for 2 h at room temperature.

T cell activation. An in-house engineered Jurkat NFAT reporter cell line was used for this study. Cells were cultured in RPMI 1640 medium (Gibco) with 10% Fetal Bovine serum (Gibco) and 1% Penicillin-Streptomycin (Gibco) at 37 °C with 5% CO2. For the activation study 100000 Jurkat cells in 100 μl full RPMI were seeded into a well of a 96 well plate per each condition and replicate. 8 μl of the functionalized liposome suspension was added, and the plate was incubated overnight at 37 °C with 5% CO2. As a positive control, Phorbol 12-myristate 13-acetate (PMA) (0.1 ng/ml final) and Ionomycin (2 ng/ml final) (both purchased from Sigma Aldrich) were added. For activation at 30 °C, the cells were incubated in an incubator with 30 °C and 5% CO2. In total, for each lipid type at least 3 biological replicates have been measured at 37 °C with at least 2 technical replicates for each biological replicate. For studies conducted at 30 °C, 3 measurements with 2 technical replicates each were conducted.

Flow cytometry. GFP positive cells were detected via flow cytometry. Samples were diluted into 1 ml flow buffer (2 mM EDTA, 1% FBS) and washed (centrifuged at 500 g for 5 min, supernatant removed, and resuspended in 300 μl flow buffer). Measurements were conducted on a BD64 LSRFortessa Cell Analyzer with sample excitation at 488 nm and GFP signal detection with the FITC filter. Events were gated for cells and single cells. The GFP positive gate was based on the αCD3 only control (no liposomes) to account for background activation from unconjugated protein, although nearly no background activation has been observed with the soluble protein. Subsequent data analysis and creation of data plots was conducted in Python.

Laurdan GP spectroscopy. Liposomes containing 0.5 mol % Laurdan were diluted to a final concentration of 400 M into the medium of choice (PBS or RPMI) and transferred into a cuvette (UV-cuvette, Brandtech). Measurements were conducted on a Cary Eclipse Fluorescence Spectrophotometer (Agilent technologies) equipped with a multicell holder, allowing for up to 4 samples to be measured at the same time. Temperature ramps were conducted with 0.5 °C/min steps and data points were acquired in 0.5 °C steps with an integration time of 1 s. The temperature was detected with a sensor placed in a cuvette filled with water allowing a feedback loop for the spectrometer to adjust its temperature during the ramp. Laurdan signal was excited at 380 nm and emission detected in two channels (I1=450 nm and 12=500 nm) in order to calculate the GP value: GP=(I1−I2)/(I1+I2).

Dynamic light scattering. Liposome size and polydispersity were measured with DLS in a 100 M dilution using a Malvern Zetasizer Nano. Results display mean values of at least two biological replicates and each replicate is the average of 3 runs with 3 reads each, in Table 2.

TABLE 2
Results of dynamic light scattering for LUVs used
for activation without functionalization.

| | z-Average | Polydispersity |
|---|---|---|
| Lipid type | (SD) in nm | Index (SD) |
| 18:1 PC | 116 (8) | 0.07 (0.01) |
| 18:1 PS | 131 (9) | 0.07 (0.01) |
| 12:0 PS | 116 (4) | 0.15 (0.01) |
| 14:0 PG | 118 (22) | 0.11 (0.02) |
| 14:0 PC | 126 (7) | 0.05 (0.03) |
| 18:0-14:0 PC | 129 (2) | 0.05 (0.01) |
| 14:0-16:0 PC | 138 (16) | 0.06 (0.08) |
| 14:0 PS | 124 (5) | 0.03 (0.01) |
| 18:1 (Delta9-Trans) PE | 168 (5) | 0.25 (0.05) |
| 14:0-18:0 PC | 136 (12) | 0.05 (0.05) |
| 16:0 PC | 129 (1) | 0.06 (0.02) |
| 16:0 PG | 125 (6) | 0.05 (0.02) |
| 17:0 PC | 142 (2) | 0.08 (0.03) |
| 16:0 PS | 145 (5) | 0.09 (0.01) |
| 18:0 PG | 151 (3) | 0.14 (0.01) |

Microscope activation study. Fluorescence images were recorded with confocal scanning microscopy on a NikonTi Eclipse microscope equipped with a confocal unit (Nikon C2 Si) and a 60× objective (Nikon Apo Lambda S 60× Oil). Activation was studied at 37 °C with a heat stage (PE100 Peltier System, Linkam). For imaging, cells were stained with 1 μM NR12S in RPMI and 1×105 cells in 130 μl full RPMI media were transferred to a μ-Slide 18 well imaging dish (#1.5, Ibidi). For activation, 20 μl of the GUV-αCD3 conjugation mixture was added to the cells. Imaging was started shortly after, as soon as the GUVs sank and started to attach to the Jurkat cells. GUVs were stained with free NR12S that was still in the cell solution. The background fluorescence of NR12S in the media was not detectable. NR12S was excited at 488 nm and emission read in two channels (I1=565 nm-595 nm and I2=600 nm-630 nm). The emission spectrum is sensitive to changes in cholesterol and membrane state and accordingly spectral shifts were detected by measuring the ratio of two characteristic wavelengths (similarly to Laurdan). To quantify emission signals, the intensities were measured over regions of interests containing a GUV or a cell. Pixel-by-pixel analysis was not used for quantification as the slight movement of vesicles lead to small displacements and the edges of the membranes did not reflect correct ratio values. Ratio images and ROI analysis was conducted in the NIS-Elements software (Nikon).

### Results and Discussion

### Lipid Nanocarrier Tₘ Affects the Activation of Jurkat Cells More so than Lipid Identity

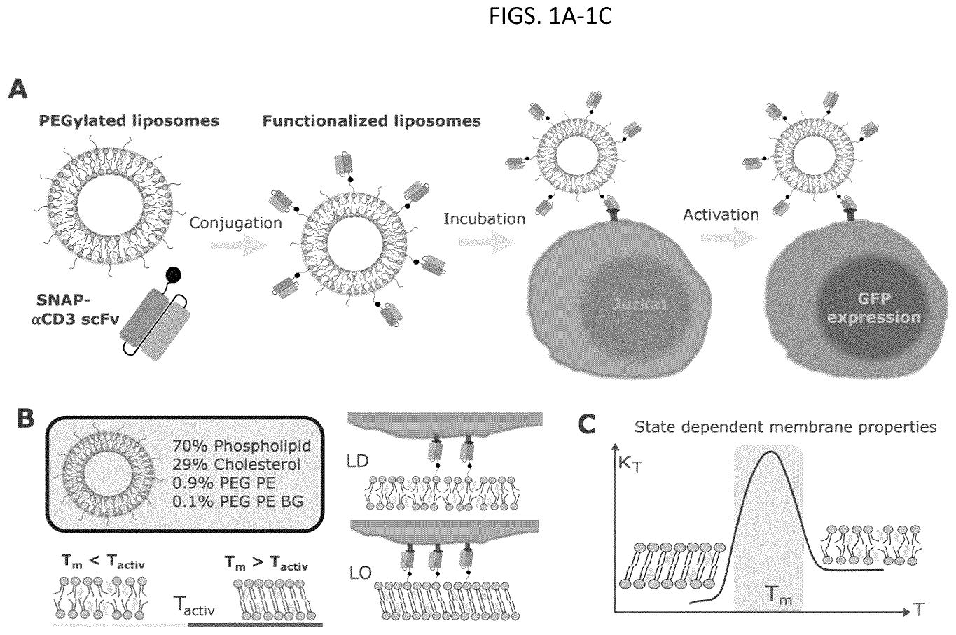

To investigate the effect of nanocarrier melting transition temperature on T cell activation, Inventors first assembled aAPCs that bind and activate T cells. aAPCs were assembled from 100 nm large unilamellar vesicles (LUVs) containing 70 mol % of a phospholipid of interest (18:1PC as a reference), 29 mol % cholesterol, 0.9 mol % 18:0 PE PEG 2000, and 0.1 mol % 18:1 PE PEG BG. The latter presents the covalent binding partner of the SNAP-tag coupled to a pegylated lipid anchor. Inventors next used a simplified one-pot workflow to assemble aAPCs by conjugating a cell free expressed SNAP-tag-αCD3 single-chain variable fragment (scFv) to LUVs. CD3 is an associated subunit of the TCR and presents a suitable target for this kind of study. An αCD3 scFv antibody fragment was designed as a fusion protein with SNAP-tag and expressed in a bacterial cell-free expression system. Upon addition of the fusion protein to liposomes containing a benzyl guanine (BG) lipid, the protein was covalently attached to liposomes and used for subsequent activation studies (FIG. 1A).

For one biological replicate, Inventors expressed one batch of protein and divided the protein between different lipid LUV samples to ensure a similar protein concentration on each LUV and so that differences in activation efficiencies relate to the lipid composition and not to protein amount. Due to varying protein yields between batches, the absolute % activation of cells varied between biological replicates. The activation levels generated by the different lipid/LUV types are noted as relative values compared to the activation of the reference sample 18:1PC within each biological replicate. In the following, if not stated otherwise, relative values in % refer to the difference in activation between the sample and the reference (DOPC liposomes):

$$( 100 \cdot \frac{{Activation}_{sample}}{{Activation}_{DOPC}} ) - 100$$

The mean of these relative activation values per lipid condition are presented in the following results. Data points represent the mean of at least 3 biological replicates.

All the lipid particles in this study were composed of the same amounts of cholesterol, PE PEG 2000, and PE PEG BG with only the type of phospholipid changed, which represents 70 mol % of the total composition. Note, since the cholesterol, PEG and BG amounts are kept constant throughout the measurements, Inventors will not explicitly refer to these components. Instead, Inventors refer to the LUV type by the variable phospholipid type alone (e.g. PE LUV). An overview of the variable phospholipids tested in this study and their properties can be found in Table 3.

TABLE 3
Overview and characteristics of lipids used in this study.

| Phospholipid | | | Net | Tₘ (°C) |
|---|---|---|---|---|
| (PL) chain length | Abbreviation | PL name | charge | of PL^{a} |
| 18:1 PC | DOPC | 1,2-dioleoyl-sn-glycero-3- | 0 | −17 |
| | | phosphocholine | | |
| 14:0 PC | DMPC | 1,2-dimyristoyl-sn-glycero-3- | 0 | 24 |
| | | phosphocholine | | |
| 18:0-14:0 PC | | 1-stearoyl-2-myristoyl-sn-glycero- | 0 | 30 |
| | | 3-phosphocholine | | |
| 14:0-16:0 PC | | 1-myristoyl-2-palmitoyl-sn- | 0 | 35 |
| | | glycero-3-phosphocholine | | |
| 14:0-18:0 PC | | 1-myristoyl-2-stearoyl-sn-glycero- | 0 | 39 |
| | | 3-phosphocholine | | |
| 16:0 PC | DPPC | 1,2-dipalmitoyl-sn-glycero-3- | 0 | 41 |
| | | phosphocholine | | |
| 17:0 PC | | 1,2-diheptadecanoyl-sn-glycero-3- | 0 | 50 |
| | | phosphocholine | | |
| 18:1 PS | DOPS | 1,2-di-(9Z-octadecenoyl)-sn- | −1 | −11 |
| | | glycero-3-phospho-L-serine | | |
| 12:0 PS | DLPS | 1,2-dilauroyl-sn-glycero-3- | −1 | 14 |
| | | phospho-L-serine | | |
| 14:0 PS | DMPS | 1,2-dimyristoyl-sn-glycero-3- | −1 | 35 |
| | | phospho-L-serine | | |
| 16:0 PS | DPPS | 1,2-dipalmitoyl-sn-glycero-3- | −1 | 51 |
| | | phospho-L-serine | | |
| 14:0 PG | DMPG | 1,2-dimyristoyl-sn-glycero-3- | −1 | 23 |
| | | phospho-(1′-rac-glycerol) | | |
| 16:0 PG | DPPG | 1,2-dipalmitoyl-sn-glycero-3- | −1 | 41 |
| | | phospho-(1′-rac-glycerol) | | |
| 18:0 PG | DSPG | 1,2-distearoyl-sn-glycero-3- | −1 | 54 |
| | | phospho-(1′-rac-glycerol) | | |
| 18:1(Δ9t)PE | | 1,2-dielaidoyl-sn-glycero-3- | 0 | 38 |
| | | phosphoethanolamine | | |

^{a}Values taken from D. Marsh, Handbook of lipid bilayers, CRC press, 2nd edn., 2013, which is incorporated by reference herein.

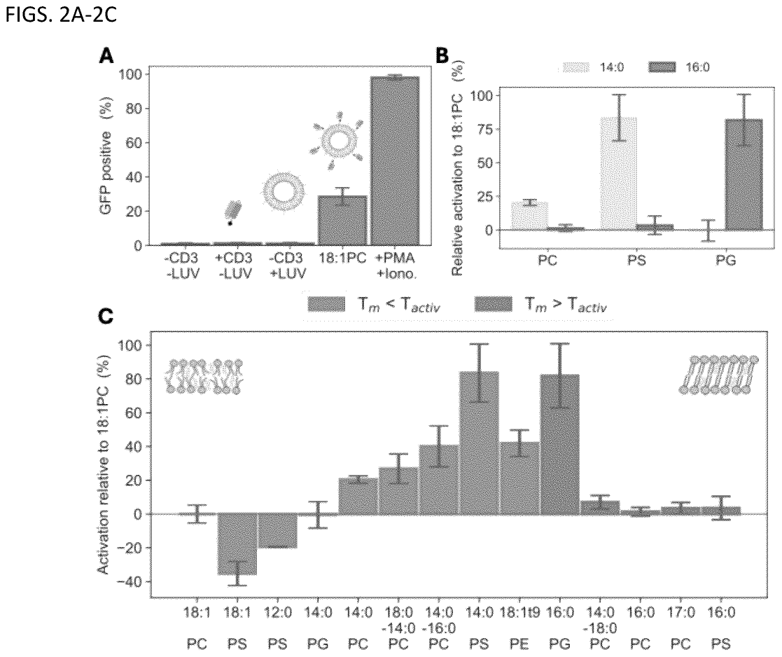

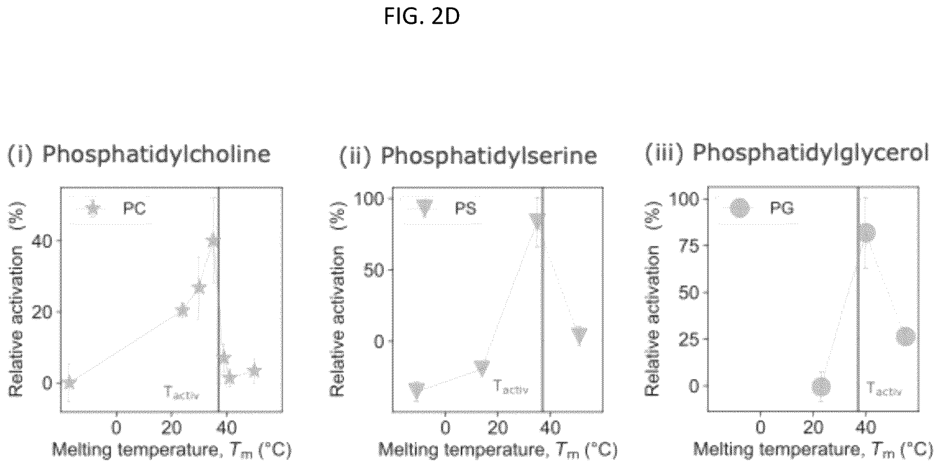

Inventors first assessed the potential of different lipids to modify nanocarrier-mediated stimulation of an immortalized line of T lymphocytes. Lipids with different features according to headgroup, charge, chain length and saturation were tested. To measure lymphocyte activation, Inventors used a Jurkat reporter cell line, in which Jurkat cell activation is reported via GFP expression. Specifically, downstream activation of the NFAT transcription factor after antigen-mediated stimulation of Jurkat cells initiates GFP expression. The percent of Jurkat cells that were activated was quantified via flow cytometry. After overnight incubation of liposome-conjugated protein with cells, the GFP signal was read out and quantified via flow cytometry. Inventors confirmed free SNAP-tag-αCD3 or blank liposomes without protein did not activate jurkat cells in the investigated concentration regime of crude cell-free product and lipid vesicles, respectively (FIG. 2A). Treatment with Ionomycin and PMA activates the cells without TCR stimulation and served as a positive control, yielding activation levels around 99% (FIG. 2A). However, due to varying yields of the αCD3-SNAP-tag protein in the cell free expression reactions, absolute activation levels for LUV-induced activation was found to differ between LUV batches and variation is reflected in the error bars shown for each experiment. Inventors first confirmed Jurkat cells could be activated with our cell-free modified aAPCs by using 18:1 PC (DOPC) LUVs that were subsequently included in each measurement. Inventors measured % of cells activated to be between 20% and 50% by DOPC LUVs.

Inventors evaluated the impact of lipid headgroup and chain length on T cell activation by assembling LUVs with the variable phospholipid containing one of three different headgroups (PC, PS, PG) and two different chain lengths (14:0 and 16:0). Inventors did not observe a correlation between a given lipid headgroup or chain length and Jurkat cell activation (FIG. 2B). In contrast, for PS LUVs, the 14:0 lipid performed better than the 16:0 lipid, whereas the trend was reversed for PG LUVs. In comparison to the reference sample (DOPC LUVs), 14:0PS and 16:0PG LUVs increase the activation significantly by +70 to 85% compared to DOPC. While 14:0PS is expected to be in the LD phase during activation, 16:0PG is expected to be in the LO phase. Surprisingly, both lipids which induced these enhanced activations have a melting transition temperature closer to the temperature at which activation studies were conducted (Tactiv=37 °C) with Tm(14:0PS)=35 °C and Tm(16:0PG)=41 °C compared to other samples (Tm(16:0PS)=54 °C and Tm(14:0PG)=23 °C). Theoretical melting temperatures reported (Table 3) of the variable phospholipid refer to pure lipid systems and do not account for neither the amount of cholesterol, PEG and BG in the liposome, nor the interaction of the LUVs with cells during activation studies. While these factors could shift the actual membrane transition point by some °C, this observation suggested a potential relationship between the lipid transition temperature and Jurkat cell activation.

To assess this potential relationship, Inventors displayed our data as a function of increasing melting temperature of the variable phospholipid in our LUVs (from left to right in FIG. 2C). Inventors observed that Jurkat cell activation relative to activation with DOPC LUVs displays a parabolic curve. LUVs containing a variable phospholipids with a Tm closest to the temperature of activation studies resulted in the highest % of activated cells. For LUVs with a variable phospholipid Tm much lower or much higher than Tactiv, the % activation decreased. This is independent of the headgroup type, chain length, saturation, or charge of the lipids Inventors tested. In general, lipids with Tm<37 °C should form disordered membranes during the activation studies at 37 °C, allowing for increased mobility of the targeting moiety on the LUV surface, which could facilitate the clustering of the TCR receptor. Rigidity or stiffness of the substrate has been reported to enhance T cell activation before but seems here to be limited to the LD phase. However, while many LD LUVs in our study seem to activate better than LO samples (Tm>37 °C), membrane order alone does not appear to explain Jurkat cell activation. The LUV offering the highest mobility is the reference sample (DOPC or 18:1 PC) as it is composed of unsaturated lipids and has the furthest distance between Tm (−17 °C) and Tactiv (37 °C). Accordingly, it should remain in the fluid LD phase during activation. Since nearly all LUVs containing a majority LD phase showed enhanced activation compared to DOPC (except for negatively charged 18:1 PS and 12:0 PS), lipid fluidity alone does not dictate Jurkat cell activation. Instead, Inventors determined that the LUV melting temperature appears to be a better predictor of Jurkat cell activation. When the data in FIG. 2C is plotted as a function of lipid Tₘ, Inventors determined LUVs with Tₘ values closest to the activation study temperature, T_{activ}, induced the most Jurkat cell activation (FIG. 2D). This trend holds regardless of lipid head group and suggests that LUV proximity to a melting transition plays an important role in Jurkat cell activation.

Jurkat Cell Activation Correlates with the Measured Melting Transitions in LUVs.

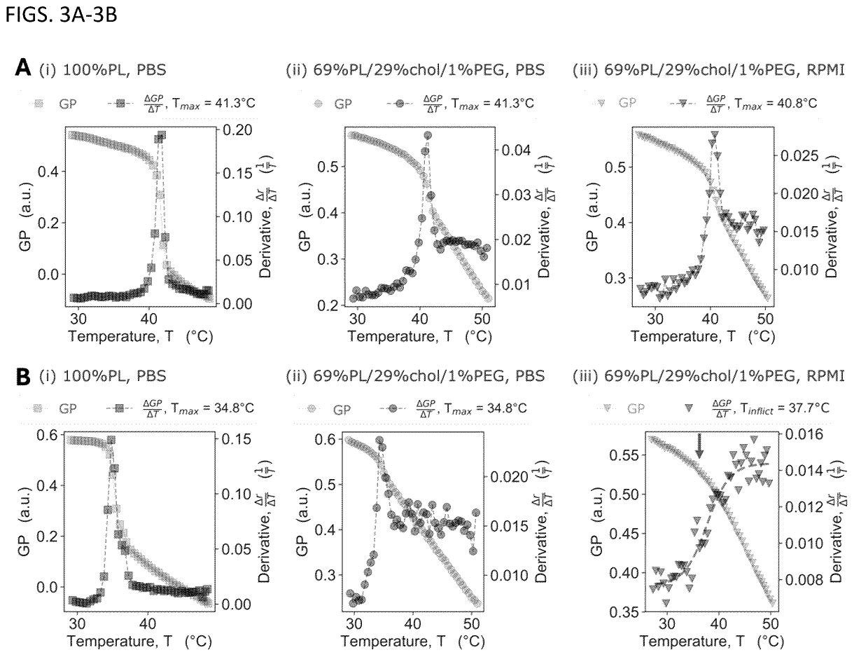

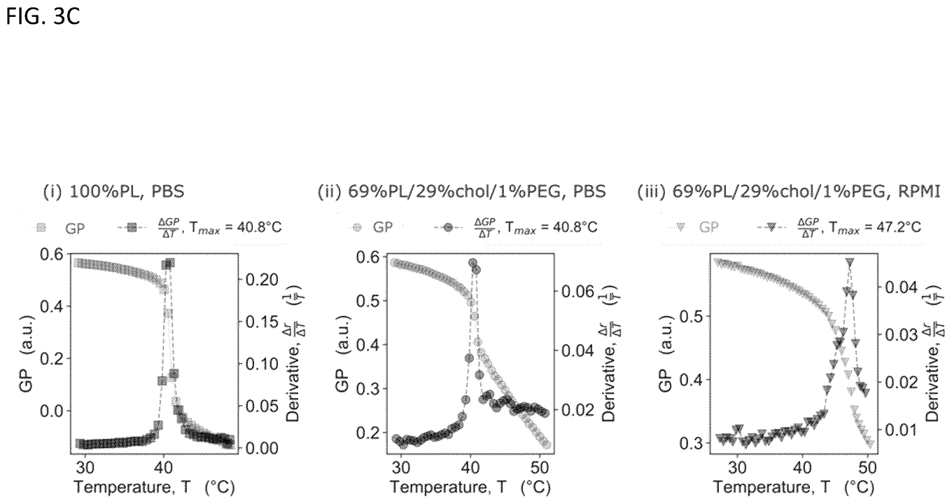

To better confirm the trend between LUV Tm and Jurkat cell activation, Inventors experimentally measured the Tm of the LUVs used in their studies. The structure and transition behavior of lipid bilayers can be affected by ions as well as interactions with components in cell culture media. To evaluate the impact of these conditions on the LUV Tm, Inventors determined LUV Tm values for LUVs expected to have a Tm close to 37 °C. To determine the Tm we measured the Laurdan generalized polarization (GP) of LUVs as a function of temperature (FIG. 3). Laurdan is a fluorescent dye displaying a shift in emission spectrum between two phase states due to its solvatochrome character. The GP value is determined by measuring Laurdan emission intensity at two characteristic wavelengths (I1=450 nm and I2=500 nm with GP=(I1−I2)/(I1+I2)). The GP shifts nonlinearly when a membrane in which it is integrated undergoes a phase transition, providing an experimental readout of the melting temperature of LUVs in our experimental conditions.

Inventors measured the melting transition temperature of LUVs in three conditions: 100% phospholipid in PBS, 70% phospholipid/29% cholesterol\1% PEG in PBS and 70% phospholipid/29% cholesterol\1% PEG in full RPMI cell media. FIG. 3 shows GP measurements as functions of temperature for 16:0PG (A), 14:0PS (B) and 16:0PC (C) LUVs for these conditions. A nonlinearity in the GP signal as function of temperature represents a transition between ordered and fluid phase. This nonlinearity is visible as a maximum in the pointwise derivative of GP with respect to temperature and occurs at the temperature with greatest GP shift. For pure phospholipid liposomes, the measured Tₘ is in good agreement with the literature values (16:0PC: Tmeas=41.8 °C and Ttheory=41 °C; 14:0PS: Tmeas=34.8 °C and Ttheory=35 °C; 16:0PG: Tmeas=40.8 °C and Ttheory=41 °C). With the addition of cholesterol and PEGylated lipids to the LUVs, the transition gets less pronounced, which is typically observed with cholesterol addition; however, the melting temperature does not shift for all LUV types tested (FIG. 3 middle panel). In RPMI media, the melting of 16:0PC LUVs is not affected by the change in environment, whereas the 16:0PG displays a strong increase in the transition temperature by ~6 °C. PG is highly susceptible to divalent ions which probably interferes with its transition behavior and membrane properties. For 14:0PS LUVs, the transition is indicated as a point where the slope of the GP function changes significantly and a transition in the structural arrangement of the lipids happens. The inflection point of the derivative is at Tinflect=37.7 °C and is increased compared to the transition temperature in PBS. Overall, while Inventors observed some variations in the actual transition temperature of LUVs relative to the transition temperature of the major variable phospholipid, most transition temperatures were close to the values reported in Table 3.

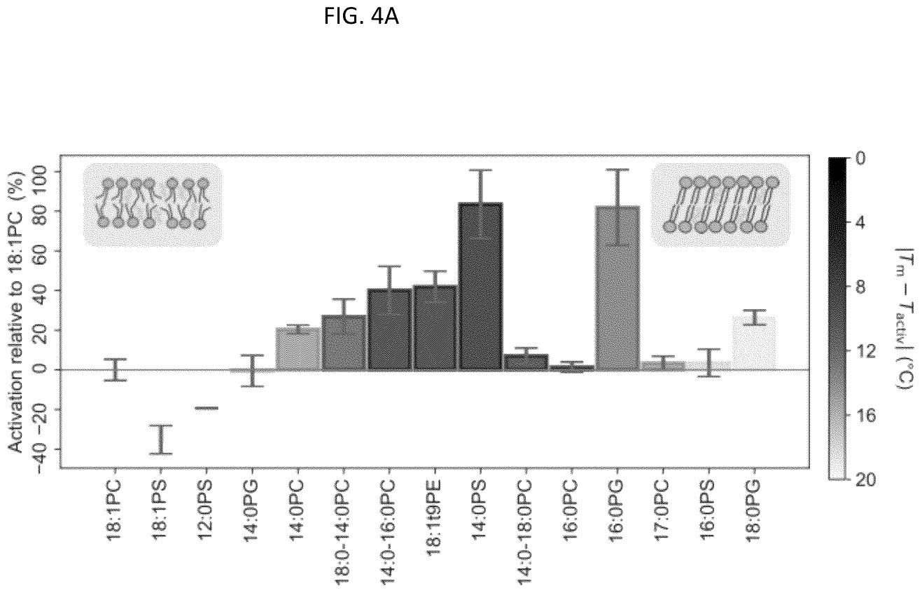

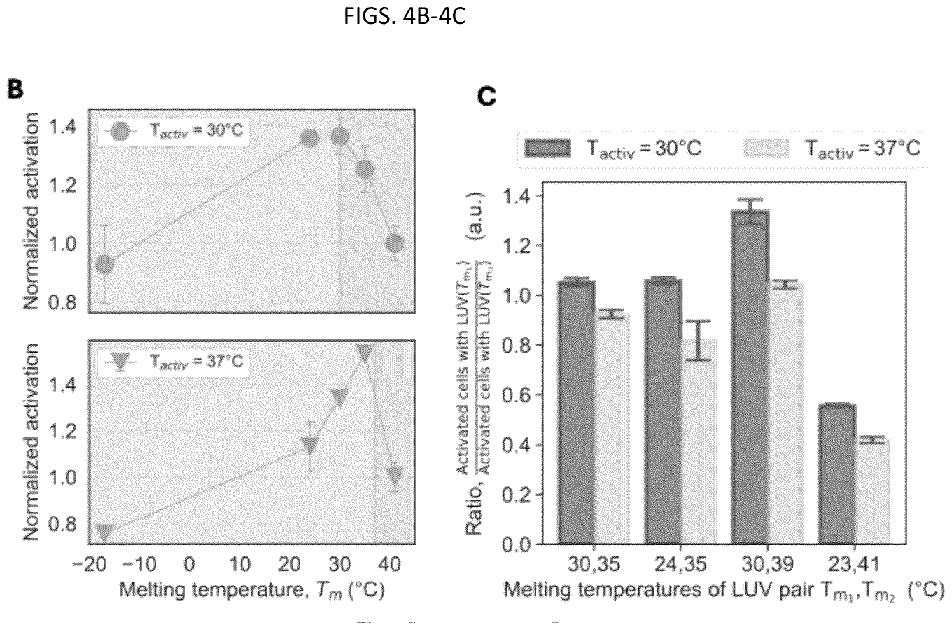

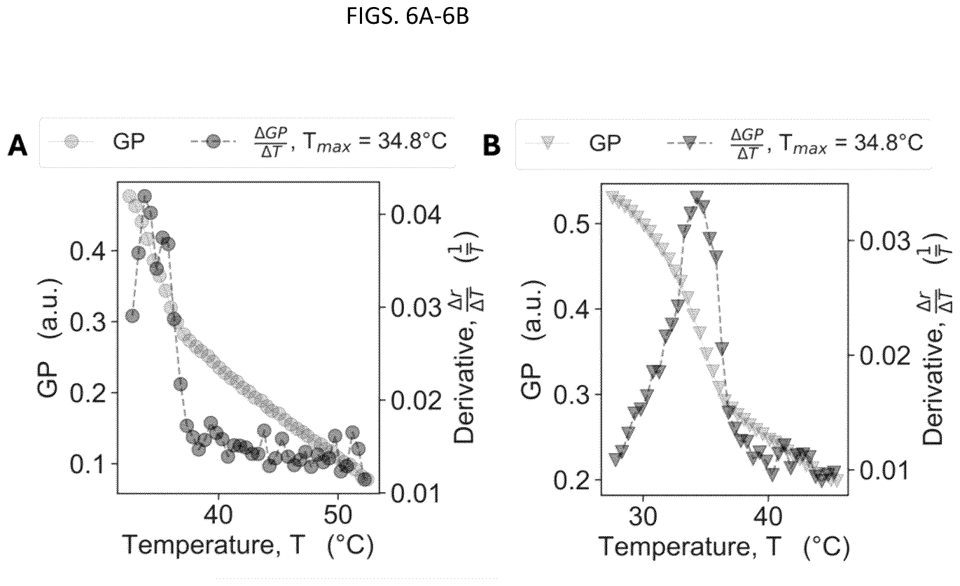

In summary, PS and PG headgroups show modified melting temperatures and shapes of the GP curve when measured in cell media as they are especially sensitive to ions. While incubation in media shifts the Tm of 14:0PS LUVs closer to 37 °C, it shifts 16:0PG LUV Tm further away presenting an anomaly from our other data showing a correlation between LUV Tm and cell activation. As expected, PC lipids are not affected by the RPMI conditions and similar trends hold for 18:1(Δ9-Trans)PE, which has a Tm we measured at 35 °C in both PBS and RPMI (FIG. 6). With these experimentally derived LUV Tm values, Inventors could evaluate the activation data again and found that 14:0PS LUVs have a higher Tm than 18:1PE LUVs (reversed from when Inventors presented Tm data according to the variable phospholipid alone in FIG. 2C). Hence, a reorganization of lipids when ordered according to their corrected transition points in the LUVs yields a cleaner correlation between the capacity to enhance activation and proximity of Tactiv to LUV Tm, with DPPG as a clear exception (FIG. 4A).

### Activation Efficiencies at a Different Temperature Tactiv.

To test the hypothesis that the melting transition of LUVs is a driving factor for enhancing Jurkat cell activation, Inventors repeated the study at an alternate temperature. Specifically, Inventors reduced the incubation temperature to 30 °C during activation. Since PC headgroups do not show significant changes in the melting temperature, these lipids are the most promising candidates to investigate the direct influence of the incubation temperature on the activation. An exemplary, direct comparison of activation levels of cells treated at the same time with the same batch of PC LUVs at 30 °C and 37 °C is shown in FIG. 4B. Indeed, 18:0-14:0PC LUVs (Tm=30 °C) resulted in the highest Jurkat cell activation at 30 °C while 14:0-16:0PC LUVs (Tm=35 °C) resulted in the highest activation at 37 °C. Inventors applied statistical analysis to this data and activation levels of lipid pairs relative to DOPC were plotted as a function of activation performance at a given temperature. This pairwise comparison (ratio of both activation levels) over n=3 biological replicates underscores the trend observed in the previous analysis (FIG. 4B). The performance of different lipids at two different activation temperatures shows a clear shift based on which lipid is closer to its transition at the given activation temperature (FIG. 4C). Adaptation of the cells towards the 30 °C incubation can be excluded as they have been grown at 37 °C prior to the overnight activation in the 30 °C and the time of incubation (16 h) is shorter than the doubling time of the Jurkats (20.7±2.2 h). These results clearly corroborate that transition-related effects in the membrane of the liposomes promote the activation process.

Due to the predicted anomalous behavior of the LUV in all properties during a melting transition, an exact mechanism responsible for the facilitated activation is merely speculation. However, nonlinear effects, like an increased amplitude of area-density fluctuations, as well as membrane undulations, are likely to increase the likelihood of antigen-receptor binding, to facilitate clustering and enhanced presentation of the antibody to the Jurkat cell surface, and to induce a mechanical signal stimulation. During synapse formation, antigens and antibodies must overcome the distance of the immunological synapse (~15 nm) in order to bind. Thermal membrane undulations of T cells membrane have been indeed proposed to initiate this binding.21 Additional transition-induced undulations of the artificial antigen presenting cell should further increase this interaction, especially for membranes in close proximity to their transition where they are expected to show maximal undulations.

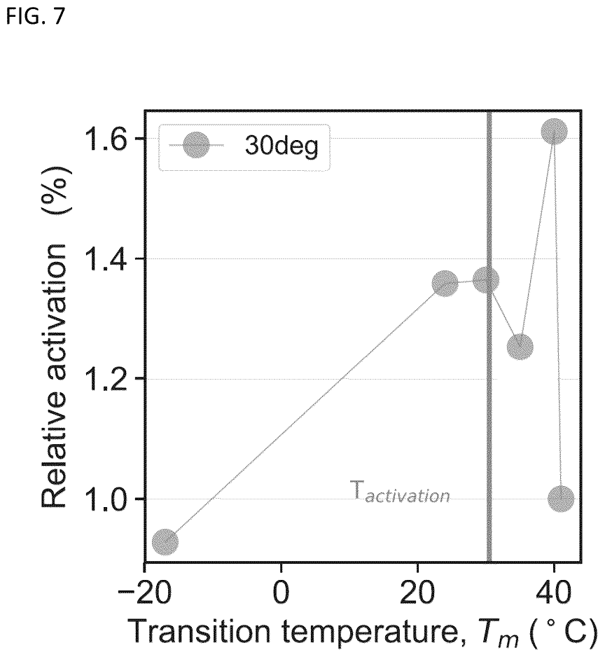

Beside PC based liposomes, PG based liposomes were also investigated at 30 °C. Surprisingly, in an overall comparison, 16:0PG LUVs were still the best activating LUV in absolute numbers compared to all tested lipids even though the difference between Tm and Tactiv has further increased (FIG. 7). The origin of the special activation potential of 16:0 PG is not known and we therefore set out to more closely examine this composition via microscopy.

Visualization of Activation with State Sensitive Dye NR12S.

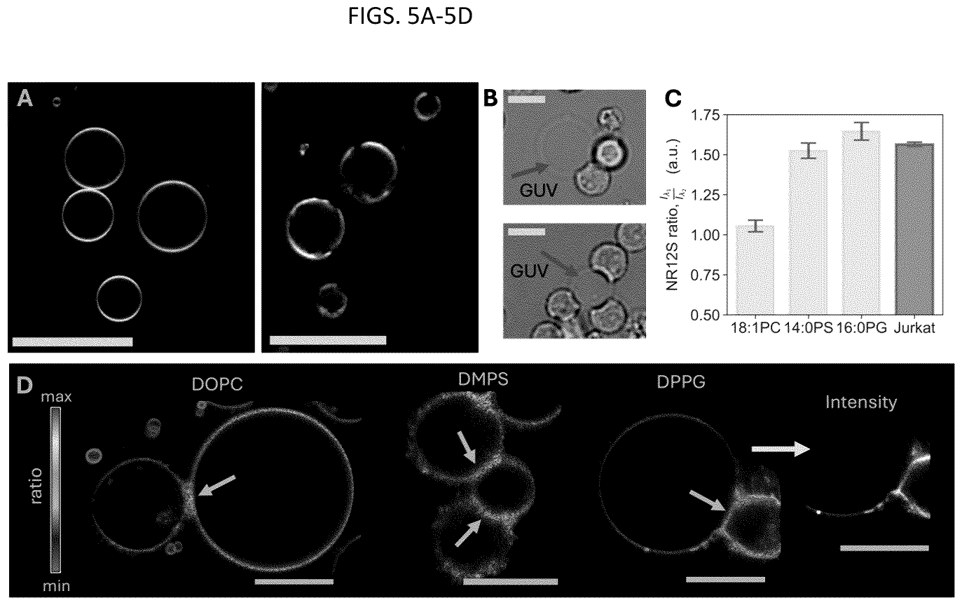

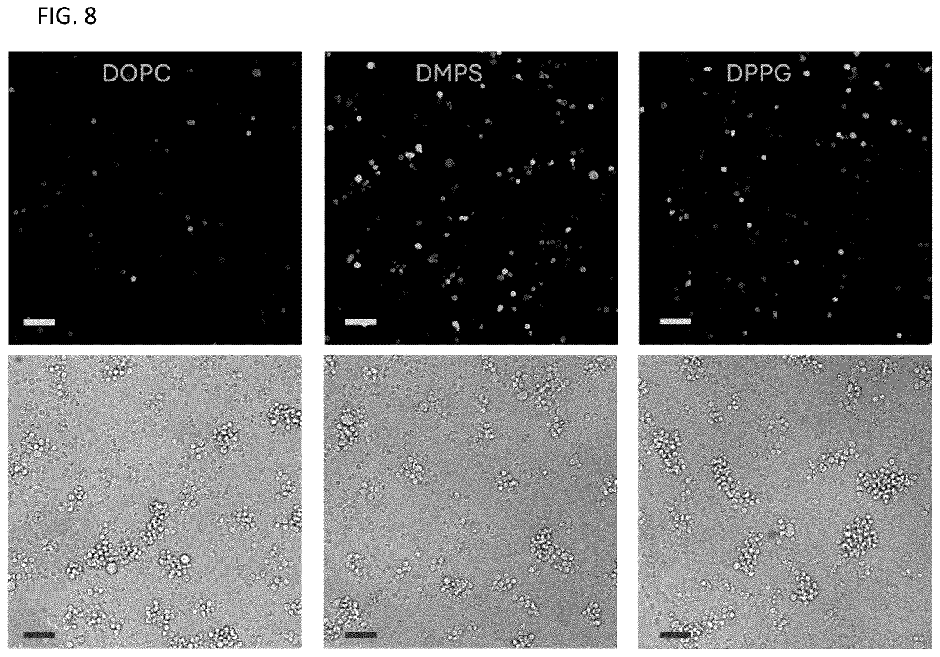

To investigate potential differences/conspicuous features of DPPG lipids and other intermediately activating lipid types in the cell activation process we used microscopy to evaluate lipid membrane-Jurkat cell interactions. Instead of LUVs (d=100 nm-200 nm), Inventors prepared giant unilamellar vesicles (GUVs) (d=5 μm-30 μm) and attached SNAP-tag-αCD3 protein similarly to the conjugation scheme used for LUVs (FIG. 5B). Inventors confirmed that GUVs prepared in this way were able to activate Jurkat cells, as GFP fluorescence was observable after an overnight incubation of Jurkats with GUV-SNAP-tag-αCD3 conjugates (FIG. 8). Cells and GUVs were stained with the state-sensitive membrane dye NR12S, and we measured the dye signal during the early activation period of the cells when the immunological synapse forms at 37 °C (30 to 60 minutes after addition of the GUVs to the cells). Inventors measured the emission intensity ratio of NR12S at two wavelengths (I1=565 nm-595 nm and I2=600 nm-630 nm, with emission ratio parameter I1/I2,). This ratio reflects the relative position of the emission spectrum, which is sensitive to cholesterol content and phase state changes in lipid and cell membranes.

Three different LUVs were compared during their antigen-mediated stimulation of the Jurkats: DOPC LUVs which serves as the standard reference in our study, DMPS LUVs, which has a melting temperature close to 37 °C, and DPPG LUVs, which displayed the highest activation potential in previous experiments. The GUVs have a similar composition to the LUVs we used for the activation studies (70 mol % PL, 29 mol % cholesterol, 0.9 mol % 18:0 PE PEG 2000, and 0.1 mol % 18:1 PE PEG BG). The NR12S signals in the different GUV types differ because of the different levels of order in these membranes at 37 °C (FIG. 5C). DOPC GUVs, far in the LD phase, display the lowest ratio, which can clearly be distinguished from the ratio of NR12S in the cell membrane at 37 °C (~1.5). DMPS is close to the optical ratio generated in the cell membrane and DPPG GUVs appear strikingly dark with a ratio around 1.6. Interestingly, small bright spots can be observed close to the cellular contact site (FIG. 5D).

The NR12S dye is known to have a reduced quantum yield (QY) in the LO phase and an even more reduced QY in the gel phase. This feature helps explain the previous result, in which DPPG GUVs displayed a lower optical ratio, and indicates DPPG membranes are in an ordered state at 37 °C. The bright dots present in local areas of the membrane, on the other hand, indicate areas of less order and suggest these areas experience higher segregation of membrane components. Indeed, a low miscibility of cholesterol in PG membranes has been reported, which can lead to the appearance of a pure cholesterol phase. With the likely segregation of cholesterol, the remaining membrane could indeed be closer to the gel phase than the LO phase, leading to additional clustering of the unsatured BG-conjugated PE anchored protein in the bright areas. Segregation of aCD3, which is attached to an unsaturated lipid, could better facilitate the Jurkat activation process. Interestingly, domains are already visible for the DPPG GUVs in 300 mosm Glucose before the dilution into RPMI media (FIG. 5A), which was not observed for DMPS and DOPC GUVs. The presence of domains can also indicate coexistence of two phases in binary mixtures. NR12S segregation was observed at room temperature as well as 37 °C, and might provide an explanation for the observed enhanced activation induced by DPPG LUVs at both tested temperatures in these studies.

To further analyze differences in the interaction of cells and different GUVs, Inventors investigated the optical signal of NR12S in the cellular contact sites, the potential immunological synapses (FIG. 5D). The ratiometric images present shifts of the color-coded NR12S emission ratio locally at the contact sites of the cells and DOPC and DMPS GUVs, which account for the local reorganization of the cell membrane in the synapse. For DOPC, the ratio of the interface between GUV and cell is increased compared to the rest of the GUV and decreased compared to the cell. For DMPS, the immunological synapse has a decreased ratio compared to the cell and GUV, while DPPG shows no change compared to the cell. The interpretation of a decrease in ratio of NR12S is not straight-forward, and either relates to a higher degree of disorder and a constant cholesterol amount or a decrease in cholesterol amount itself. For membranes in the solid gel phase, additional cholesterol decreases the order, while for membranes in the LD phase, cholesterol increases order. For cell membranes, there are several examples showing a depletion of cholesterol leads to an increase in membrane tension, which would lead to the observed local decrease in ratio, supporting reports that the immunological synapse is more ordered compared to the rest of the cell. A local accumulation of lipids with higher Tₘ and potentially local displacement of cholesterol in the Jurkat cell membrane at the site of the synapse could lead to the observed decrease in ratio and could explain the observed, local membrane state changes. At present, however, we do not have an explanation for the enhanced performance of DPPG-based vesicles.

### CONCLUSION

These results demonstrate that the interaction between T cells and substrates is not determined by the choice of targeting moiety, neither is it determined by membrane fluidity alone. Rather, the choice of the phospholipid in nanocarrier design can either support the interaction efficiency of the targeting moiety with the target cell or even suppress it as a result of the modulation of properties of the functionalized membrane interface.

LUVs residing close to their transition regime during incubation tended to activate best in this study independent of their individual features such as headgroup, chain length, saturation, or even charge. Laurdan GP measurements indicate that there is indeed still a measurable melting transition present in the tested LUV compositions albeit their relatively high cholesterol content of 29 mol %. These results suggest that phase transition related effects and the associated anomalous behavior can enhance the capacity of designed nanocarrier substrates to activate target cells. Specifically, Inventors hypothesized that as the Tm of nanocarrier membranes comes in close proximity to the temperature at which activation studies are conducted, density fluctuations in both membranes should promote local accumulations of the antibody and increased membrane undulations should facilitate antigen-receptor binding.

An exception of the observed correlation with Tm was found for 16:0PG LUVs, as this nanocarrier led to increased Jurkat activation level than that induced by 14:0PS LUVs even though its Tm was measured to be ~10 °C above Tactiv. Interestingly, 16:0PG still performs best at 30 °C incubation temperature (FIG. 7) and the activation capacity of this composition LUV appears to be independent of the proximity of its Tm to Tactiv. Domains observed in the GUVs made from the 16:0 PG lipids suggests cholesterol accumulations in which the PE anchor of the BG group would cluster too. Hence, segregation-related clustering could explain the improved activation capabilities for the temperatures at 30 °C and 37 °C opening up another potential interesting feature for the design of nanocarriers. These findings set the stage for future investigations exploring the design of LUVs and the role of both LUV Tm and domain-segregation of targeting molecules.

1. A method of activating a cell with a lipid nanocarrier, the method comprising:
determining a melting transition temperature of the lipid nanocarrier;
selecting an incubation temperature, wherein the melting transition temperature and the incubation temperature differ by no greater than about 10 °C; and
contacting the cell with the lipid nanocarrier at the incubation temperature.

2. The method of claim 1, wherein the melting transition temperature and the incubation temperature differ by no greater than about 5.0 °C.

3. The method of claim 1, wherein the melting transition temperature and the incubation temperature differ by no greater than about 1.0 °C.

4. The method of claim 1, wherein the melting transition temperature and the incubation temperature are substantially the same.

5. The method of claim 1, wherein the lipid nanocarrier comprises a lipid selected from the group consisting of 1,2-dioleoyl-sn-glycero-3-phosphocholine (DOPC), 1,2-di-(9Z-octadecenoyl)-sn-glycero-3-phospho-L-serine (sodium salt) (18:1PS;DOPS), 1,2-dilauroyl-sn-glycero-3-phospho-L-serine (sodium salt) (12:0PS; DLPS), 1,2-dimyristoyl-sn-glycero-3-phospho-(1′-rac-glycerol) (sodium salt) (14:0PG; DMPG), 1,2-dimyristoyl-sn-glycero-3-phosphocholine (14:0PC; DMPC), 1-stearoyl-2-myristoyl-sn-glycero-3-phosphocholine (18:0-14:0PC), 1-myristoyl-2-palmitoyl-sn-glycero-3-phosphocholine (14:0-16:0PC), 1,2-dimyristoyl-sn-glycero-3-phospho-L-serine (sodium salt) (14:0PS; DMPS), 1,2-dielaidoyl-sn-glycero-3-phosphoethanolamine (18:1(Δ9-Trans)PE), 1-myristoyl-2-stearoyl-sn-glycero-3-phosphocholine (14:0-18:0PC), 1,2-dipalmitoyl-sn-glycero-3-phospho-(1′-rac-glycerol) (sodium salt) (14:0 PG; DPPG), 1,2-dipalmitoyl-sn-glycero-3-phosphocholine (16:0PC;DPPC), 1,2-diheptadecanoyl-sn-glycero-3-phosphocholine (17:0 PC), 1,2-dipalmitoyl-sn-glycero-3-phospho-L-serine (sodium salt) (16:0 PS; DPPS), 1,2-distearoyl-sn-glycero-3-phospho-(1′-rac-glycerol) (sodium salt) (18:0 PG, DSPG), and any combinations thereof.

6. The method of claim 1, wherein the lipid nanocarrier comprises about 70 mol % of a lipid.

7. The method of claim 1, wherein the lipid nanocarrier comprises cholesterol and 1,2-distearoyl-sn-glycero-3-phosphoethanolamine-N-[methoxy(polyethylene glycol)-2000](ammonium salt) (18:0 PE PEG 2000).

8. The method of claim 1, wherein the lipid nanocarrier comprises a lipid anchor.

9. The method of claim 8, wherein the lipid anchor comprises polyethylene glycol (PEG).

10. The method of claim 9, wherein the lipid anchor comprises 1,2-dioleoyl-sn-glycero-3-phosphoethanolamine-N-[benzylguanine(polyethylene glycol)-2000](18:1 PE PEG BG).

11. The method of claim 8, wherein the lipid anchor is functionalized with at least one surface tag.

12. The method of claim 11, wherein the at least one surface tag is selected from the group consisting of an antibody, a protein, a co-stimulatory ligand, a cytokine, an antigen, and any combinations thereof.

13. The method of claim 11, wherein the surface tag comprises αCD3.

14. The method of claim 1, wherein the lipid nanocarrier comprises a liposome.

15. The method of claim 1, wherein the cell is a T-cell.

16. The method of claim 1, wherein the incubation temperature ranges from about 4 °C to about 65 °C.

17. The method of claim 1, wherein the incubation temperature ranges from about 20 °C to about 55 °C.

18. A method of treating a T-cell related disorder in a subject in need, the method comprising:
selecting a lipid nanocarrier comprising a melting transition temperature, wherein the melting transition temperature and the subject's body temperature differ by no greater than 10 °C; and
administering an effective amount of the lipid nanocarrier to the subject.

19. The method of claim 18, further comprising administering an effective amount of a chimeric antigen receptor (CAR) T-cell to the subject in need.

20. The method of claim 18, further comprising determining the melting transition temperature of the lipid nanocarrier prior to the step of selecting the lipid nanocarrier.
